(19) United States
(12) Patent Application Publication
(10) Pub. No.: US 2026/0086019 A1
(43) Pub. Date: Mar. 26, 2026

(54) METHOD FOR CHARACTERISING THE DEFORMABILITY OF CELLS OR A PORTION OF CELLS IN A CELL SAMPLE

(71) Applicants: Ecole Polytechnique, Palaiseau (FR); Centre National De La Recherche Scientifique, Paris (FR)

(72) Inventors: Abdul BARAKAT, Palaiseau (FR); Claire LECLECH, Palaiseau (FR); Bettina ROELLINGER, Palaiseau (FR)

(21) Appl. No.: 19/109,882

(22) PCT Filed: Sep. 8, 2023
(86) PCT No.: PCT/EP2023/074782

§ 371 Date: Dec. 15, 2025

(30) Foreign Application Priority Data
Sep. 8, 2022 (FR) FR2209022

(51) Int. Cl. G01N 15/14; G01N 33/483

(52) U.S. Cl. CPC G01N 15/1484; G01N 33/4833; G01N 2015/1495

(57) ABSTRACT

The invention relates to a method for characterising the deformability of cells or a portion of cells in a sell sample, the method comprising: culturing the cells on a microstructured plate having microgrooves with a predetermined width and depth for at least partially engaging the nuclei of the cells in one or more microgrooves, at least one portion of the surface of the microgrooves being a cell adhesion surface; measuring a fluorescence signal of the nuclei, which nuclei are pre-treated so as to emit fluorescence radiation; on the basis of the fluorescence signal measured for each nucleus, determining a fluorescence intensity profile and at least one morphological parameter of the nucleus; on the basis of the fluorescence intensity profile and the at least one morphological parameter, determining a deformation class of the nucleus in the depth of the microgrooves.

The present invention relates to a process for characterizing the deformability of cells or a portion of cells, in particular cell nuclei, in a cell sample. The present invention also relates to a method for diagnosing a disease state in an individual, and a method for screening a candidate compound for the treatment and/or prevention of a disease state.

### TECHNICAL FIELD

Generally speaking, the mechanical properties of cell nuclei are nowadays considered to be important biomarkers in numerous diseases. Currently, the most commonly used laboratory experimental systems to test these mechanical properties, such as atomic force microscopy, micropipette aspiration or microrheometry, are low-throughput, technically complex and/or costly from both an equipment and time perspective. Novel microfluidic experimental systems using circulation of cells in channels have been developed. They enable higher throughputs, but remain complex.

It is known from the article by Antmen E et al: Amplification of nuclear deformation of breast cancer cells by seeding on micropatterned surfaces to better distinguish their malignancies. Colloids Surf B Biointerfaces. 2019 Nov. 1:183:110402. doi: 10.1016/j.colsurfb.2019.110402. Epub 2019 Jul. 30. PMID: 31398621 to deposit cancer cells, the nuclei of which have been made fluorescent, on a plate having protruding microreliefs in the form of mutually parallel pillars of sizes smaller than the cells, and to determine, by analyzing fluorescence images of the plate, certain morphological parameters of the nuclei associated with their deformation on the plate due to the pillars, in order to deduce therefrom whether the cells are metastatic or healthy. Such fluorescence image analysis is complex, due to the variety of shapes that the cell nuclei can adopt on such a plate.

It is known from the article by Alvarez-Elizondo M B et al. Micropatterned topographies reveal measurable differences between cancer and benign cells. Med Eng Phys. 2020 January: 75:5-12. doi: 10.1016/j.medengphy.2019.11.004. Epub 2019 Nov. 25. PMID: 31780301 to deposit cancer cells having a fluorescent nucleus on a plate having parallel microgrooves of a size smaller than the cells, and to observe the morphology in the plane of the plate and the orientation of the cells and of their nuclei in the direction of the microgrooves, in order to deduce therefrom whether the cells are metastatic or healthy. Such an observation is made by adjusting a contour ellipse for each nucleus and by determining the long and short axes thereof, the area thereof, the eccentricity thereof and the orientation thereof in the microgroove for a highly specific plate topography which must be precisely determined beforehand. However, no information regarding deformation in the depth is studied.

There is a need for a simple, robust and high-throughput process for characterizing the mechanical deformation properties of cell nuclei which is reliable, easy to implement in clinical practice and relatively inexpensive, in particular in order to make it possible to carry out quick and reliable functional tests, making it possible in particular to detect a disease or non-disease state in a cell sample, for example with the aim of making a diagnosis or screening a compound of interest.

### DISCLOSURE OF THE INVENTION

The invention meets this need by means of a process for characterizing the deformability of cells or a portion of cells in a cell sample, said cells each comprising a body and a nucleus, the process comprising:
culturing said cells on a microstructured plate which has, at the surface, a plurality of microgrooves, the microgrooves being of a predetermined width, spacing and depth so as to enable the at least partial engagement of the nucleus of at least one of said cells in one or more of the microgrooves, at least part of the surface of the microgrooves being an adhesion surface for the cells,
measuring, by microscopy, a fluorescence signal of the nucleus of said cells, the nucleus of said cells having been treated beforehand to emit fluorescence radiation,
based on the fluorescence signal measured for each nucleus, determining a fluorescence intensity profile for each nucleus along at least one axis of said nucleus, and at least one morphological parameter of said nucleus,
based on the fluorescence intensity profile and on at least one morphological parameter determined for each nucleus, determining a deformation class of said nucleus in the depth of one or more microgrooves.

As mentioned previously, the nuclei of the cells can deform on a microstructured adherent surface. The deformation of the nuclei depends on mechanical and biological characteristics of the cell, for example the rigidity thereof or the contractility thereof. Such deformation characteristics may be different depending on the physiological or disease state of a cell type. Studying the deformation characteristics of cell nuclei from a sample can therefore make it possible to determine a biological state, in particular a disease state, of a cell sample.

The presence of microgrooves on the microstructured plate with an adhesion surface makes it possible to generate a particular deformation stress on the cells, controlled by the dimensions of the microgrooves on the plate. Indeed, the adhesion of cells to the walls of the microgrooves leads to the deformation of the nuclei according to several known geometries and/or configurations, possibly going so far as to completely trap the nuclei in the depth of the microgrooves. This makes it possible to determine different deformation classes of the nuclei. Determining a deformation class for each nucleus can make it possible to compare the percentages of cells which are classified in the deformation classes compared to a reference sample, in order to deduce a biological characteristic therefrom.

The comparative study of a statistical intensity or morphometric variable, in particular of a distribution of a parameter of the fluorescence intensity profile or a distribution of a morphological parameter, of the nuclei of the cells in the sample which are classified in at least one deformation class, can make it possible to have greater reliability in the determination of a biological characteristic of the sample than the comparative study of said statistical intensity or morphometric variable for all the nuclei of the cells in the sample.

The process is an ex vivo or extemporaneous process.

The process is non-therapeutic per se.

### Microstructured Plate

The microstructured plate preferably comprises a transparent support, in particular made of glass, and a microstructured polymer structure having microgrooves at the surface, in particular made of polydimethylsiloxane (PDMS).

The microgrooves are preferably mutually parallel.

The microgrooves are preferably of a predetermined width and depth so as to give rise to the deformation of a portion of the cells in the direction of the depth of the microgrooves.

The width, depth and spacing of the microgrooves can be chosen on the basis of at least one physical parameter of at least some cells of the cell type, in particular the dimension of said cells, the dimension of the nucleus of said cells, and/or a biophysical characteristic of said cells, in particular the rigidity, adhesion strength or contractility of said cells. The width, depth and spacing of the microgrooves can be chosen to have a mean percentage of nuclei entirely trapped in the microgrooves on the basis of the predetermined type of cells in the sample.

The mean percentage of nuclei of healthy or abnormal cells of said cell type entirely trapped in the microgrooves can be between 5% and 95%, better still between 10% and 90%.

The width, depth and spacing of the microgrooves is preferably between 30 and 60% of the length of the mean minor axis of the nucleus of the non-deformed cells.

“Length of the mean minor axis” means the mean length of the small axis of an ellipse, adjusted to the shape of the nucleus of non-deformed cells.

The width of the microgrooves is predetermined for a cell type and varies on the basis of the biological characteristics of the cell types being studied. Generally speaking, the width is greater than or equal to 3 μm and/or less than or equal to 10 μm.

The depth of the microgrooves is predetermined for a cell type and varies on the basis of the biological characteristics of the cell types being studied. Generally speaking the depth is greater than or equal to 4 μm and/or less than or equal to 10 μm. For muscle cells, the depth can be between 4 and 5 μm in order to obtain a sufficiently high content of nuclei in the microgrooves. In the case of breast epithelial cells, the depth can be between 7 and 9 μm, for example substantially equal to 8 μm. The microgrooves can all be of a substantially constant depth over the entire length thereof.

The microgrooves can have a spacing between them, measured between adjacent edges, of greater than or equal to 3 μm and/or less than or equal to 10 μm.

The microgrooves can all be of a substantially constant width over the entire length thereof. The microgrooves can all be of a substantially constant depth over the entire length thereof.

The microgrooves can all be of a substantially constant spacing over the entire length thereof.

As a variant, the microstructured plate can comprise distinct zones, each comprising microgrooves of different widths and/or spacings.

The depth may be fixed or variable on a microstructured plate and/or along a microgroove.

At least part of the inner surface of the microgrooves, in particular the side walls and/or the bottom of the microgrooves, preferably the whole surface of the microstructured plate, can be coated with an adhesion coating, in particular a cell adhesion protein, for example fibronectin, collagen, laminin or gelatin.

### Cell Sample

The cells can be adherent cells, for example muscle cells, endothelial cells, stem cells, preferably non-embryonic or non-human stem cells, epithelial cells, nerve cells, bone cells, adipose cells, podocytes, cancer cells or a mixture of such cells.

The process can comprise a step of fixing the cells to the microstructured plate by adding an alcohol compound, in particular methanol, or an aldehyde compound, in particular paraformaldehyde. The step of fixing the cells can be carried out at least one hour after the sample is deposited on the microstructured plate. Such a length of time makes it possible to give the cells time to deform before they are fixed, in order to have a result indicative of the deformation of the cells in the sample.

The process can comprise treating the cell sample in order for the nuclei to emit the fluorescent radiation mentioned above. This step can take place before or after the cells of the sample are placed on the microstructured plate, preferably after the cells of the sample are placed on the microstructured plate, better still after the cells are fixed to the microstructured plate.

The nucleus of the cells of the sample can be labeled before or after fixation, using a fluorescent nuclear label, in particular a Hoechst stain or DAPI.

As a variant, the treatment of the cells in order for the nucleus to emit fluorescent radiation can comprise transduction of the cells with a plasmid encoding a nuclear protein fused to a fluorescent protein.

The cell sample can be of human, animal or plant origin.

The cell sample is preferably configured so that the cell density of the cell sample at the moment of the microscopy measurement avoids cell confluence, in particular so that the cell confluence is less than or equal to 60%. For example, for muscle, endothelial or epithelial cells, the cell density of the sample deposited on the microstructured plate can be greater than or equal to 10 000 cells/cm² and/or less than or equal to 50 000 cells/cm².

### Microscopy Measurement

The microscopy measurement can comprise the acquisition of a fluorescence image of the surface of the microstructured plate. The fluorescence image obtained can be a view from above or below by transparency of the microstructured plate, in particular obtained by epifluorescence.

As a variant or in combination, the microscopy measurement can comprise acquiring fluorescence images in a plane and/or a plurality of planes transverse to the microgrooves, in particular by confocal microscopy.

The microscopy measurement can comprise the detection of the fluorescence signal emitted by the nucleus of each cell on the acquired fluorescence image(s).

### Image Analysis

The process, in particular the step of determining the fluorescence intensity profile and the morphological parameter(s), can comprise detecting nuclei on the image, and also the contour of said nuclei, based on the fluorescence signal emitted by the nucleus, which is in particular visible on the acquired fluorescence image(s), and the morphometric analysis of said shape of the nucleus.

All of the steps of determining the fluorescence intensity profile and the at least one morphological parameter, and determining the deformation class, can be carried out automatically, in particular by a processor running software for processing the fluorescence images obtained by the microscopy measurement. The processor can comprise automatic or deep learning software.

### Determination of Fluorescence Intensity Profile

The fluorescence intensity profile can be determined based on the fluorescence signal, in particular on the acquired fluorescence image, perpendicularly to the axis of extension of the microgrooves, preferably in a substantially mid-plane of said nucleus.

### Determination of Morphological Parameter

The at least one morphological parameter of the nucleus of the cell can be chosen from the circularity, roundness, solidity, aspect ratio, and/or the ratio of elliptic Fourier coefficients of the nucleus, and/or the negative mean curvature of the contour of the nucleus.

“Negative mean curvature” means the mean of the concave curvatures of the contour of the nucleus.

The morphological parameter(s) can be selected, in particular by prior analyses on reference samples, so as to be one or more relevant parameters for discriminating between different biological characteristics of the sample, in particular between a disease characteristic and a healthy characteristic. The morphological parameter(s) are selected such that, for a distribution of the or each morphological parameter, a statistical difference is established between samples having different biological characteristics, in particular healthy and diseased.

### Determination of the Deformation Class

The process preferably comprises, based on the fluorescence intensity profile and on at least one morphological parameter for each nucleus, determining the deformation class of said nucleus in the depth of one or more microgrooves from at least three predetermined deformation classes which correspond, respectively, to:
a) a freely suspended nucleus, corresponding for example to a substantially flat fluorescence intensity profile or one which is devoid of major fluorescence intensity peaks, associated with one or more morphological parameters characteristic of a nucleus contour that is not very deformed, and/or of a low tortuosity of the nucleus contour, for example a high degree of circularity, roundness, solidity and/or ratio of elliptic Fourier coefficients, in particular greater than or equal to a respective predetermined threshold value, and/or a low aspect ratio and/or negative mean curvature, in particular less than or equal to a respective predetermined threshold value,
b) a deformed nucleus, extending into at least two adjacent microgrooves, corresponding for example to a fluorescence intensity profile having at least two main peaks, associated with at least one morphological parameter characterizing significant tortuosity of the contour, in particular low solidity compared to a predetermined threshold value, and/or a low ratio of elliptic Fourier coefficients compared to a predetermined threshold value, and/or a high negative mean curvature compared to a predetermined threshold value, and/or low circularity and/or roundness compared to a predetermined threshold value,
c) a trapped nucleus, in particular entirely inserted in a microgroove, corresponding for example to a fluorescence intensity profile having a main peak, associated with at least one morphological parameter characterizing elongation of the nucleus, in particular low circularity and/or roundness compared to a predetermined threshold value, and/or a high aspect ratio compared to a predetermined threshold value.

The process can comprise comparing at least one statistical characteristic of at least one obtained deformation class of the nuclei of the cells from the sample with the same characteristic for a reference sample. The statistical characteristic can be the proportion of cells in the cell sample in at one deformation class, and/or a statistical or morphometric variable of the nuclei of the cells which are classified in at least one deformation class.

The process can comprise determining the proportion of cells in the cell sample in at least one of the deformation classes, in particular the class of trapped and/or deformed nuclei. The process can comprise determining a biological characteristic of the cell sample by comparing the proportion of cells in the cell sample which are in the or each deformation class with a proportion of cells in said at least one deformation class in a reference sample of the same cell type, the biological characteristic of the reference sample being known.

The process can comprise determining a statistical distribution of the or at least one of the morphological parameters of the nuclei of the cells from the sample which are classified in one of the deformation classes, in particular for the class of deformed nuclei and/or the class of trapped nuclei. The process then preferentially comprises comparing a variable of said statistical distribution of the sample with the same variable of statistical distribution of the morphological parameter of cells which are classified in said same deformation class(es) in a reference sample for which the biological characteristic is known. The inventors have demonstrated that limiting the morphological comparison to the cells of a specific deformation class in a sample makes it possible to achieve more precise biological characterization of the sample than when all the cells in the sample are used.

The biological characteristic can be the diseased or non-diseased nature of the cell sample.

The process can comprise a prior step of determining the deformation class for which the proportion of cells which are classified in said deformation class, measured for reference samples, enables the best possible discrimination between at least two different populations of biological characteristics.

As a variant or in addition, the process can comprise a prior step of determining a combination of one or more morphological parameters of the nuclei, and one or more deformation classes for which a variable of statistical distribution of the morphological parameter(s) of the nuclei which are classified in the deformation class(es) enables the best possible discrimination between two reference populations having difference biological characteristics.

This or these prior determination steps can comprise one or more statistical tests for comparing the samples.

### Diagnostic Method

The invention also meets this need by means of a method for diagnosing a disease state in an individual, the method comprising at least the following steps:
a) culturing a sample of a type of cells isolated from said individual, the cells of the sample comprising a body and a nucleus and being cultured on a microstructured plate having, at the surface, a plurality of microgrooves, at least part of the surface of the microgrooves being an adhesion surface for the cells, the microgrooves being of a predetermined width and depth so as to enable the at least partial engagement of the nucleus of at least one of said cells in one or more of the microgrooves,
b) measuring, by microscopy, a fluorescence signal of the nuclei of the cells from the sample, the nuclei of the cells having been configured beforehand to emit fluorescence radiation,
c) based on the fluorescence signal measured for each nucleus, determining a fluorescence intensity profile for each nucleus along at least one axis of said nucleus, and at least one morphological parameter of said nucleus,
d) based on the fluorescence intensity profile and on at least one morphological parameter determined for each nucleus, determining a deformation class of said nucleus in the depth of one or more microgrooves,
e) comparing at least one characteristic of at least one deformation class of the nuclei of the cells from the sample, obtained in step d), with the same characteristic for a reference sample, in order to reach a conclusion therefrom regarding the disease state of the individual.

The characteristics described previously in relation to the process for characterizing the deformability of a cell or a portion of a cell apply to this diagnostic method in combination with, or independently of, one another, and independently of the process for characterizing the deformability of a cell or a portion of a cell.

### Screening Method

The invention also meets this need by means of a method for screening a candidate compound for the treatment and/or prevention of a disease state, the method comprising at least the following steps:
a) in vitro culture of a first sample of a cell type representative of a disease in the absence of the candidate compound,
b) in vitro culture of a second sample of said cell type representative of said disease in the presence of the candidate compound,
   the cells of the first and second samples comprising a body and a nucleus and being cultured on a first and a second identical microstructured plate each having, at the surface, a plurality of microgrooves, at least part of the surface of the microgrooves being an adhesion surface for said cells, the microgrooves being of a predetermined width and depth so as to enable the at least partial engagement of the nucleus of at least one of said cells in one or more of the microgrooves,
c) measuring, by microscopy, a fluorescence signal of the nuclei of the cells from the first and second samples, the nuclei of the cells from the first and second samples having been configured beforehand to emit fluorescence radiation,
d) determining fluorescence intensity profiles along at least one axis of the nucleus, and at least one morphological parameter of the nucleus of each cell from the first and second samples, based on the respective measured fluorescence signals,
e) determining deformation classes of the nucleus of each cell from the first and second samples in the direction of the depth of the microgrooves, based on the determined fluorescence intensity profile and on at least one determined morphological parameter, and
f) comparing at least one characteristic of the first sample in at least one deformation class of the nuclei of the cells from the first sample with the same characteristic for the second sample, the observation of a difference between said characteristics of the first and second sample being indicative of the efficacy of the candidate compound with respect to said disease.

The process can comprise the culture of a third sample of said cell type which is considered to be healthy in the absence of the candidate compound; measuring, by microscopy, a fluorescence signal of the nuclei of the cells from the third sample, the nuclei of the cells of the third sample having been configured beforehand to emit fluorescence radiation; determining the fluorescence intensity profile along at least one axis of the nucleus, and at least one morphological parameter of the nucleus of each cell from the third sample, based on the respective measured fluorescence signals; determining deformation classes of the nucleus of each cell from the third sample in the direction of the depth of the microgrooves, based on the determined fluorescence intensity profile and on at least one determined morphological parameter; and comparing at least one characteristic of the first sample and/or of the second sample in at least one deformation class of the nuclei of the cells from the first sample and/or from the second sample with the same characteristic for the third sample, the observation of a difference between said characteristics of the first and third sample and/or a similarity between said characteristics of the second and third sample being indicative of the efficacy of the candidate compound with respect to said disease.

The characteristics described previously in relation to the process for characterizing the deformability of a cell or a portion of a cell apply to this screening method in combination with, or independently of, one another, and independently of the process for characterizing the deformability of a cell or a portion of a cell.

Preferably, with the characteristic being the proportion of cells in the samples which are in at least one of the deformation classes, in particular the class of trapped nuclei, the observation of a statistical difference between the first and second sample, and/or the observation of a non-significant statistical difference between the healthy sample and the sample in the presence of the candidate compound and of a significant statistical difference between the healthy sample and the sample without the candidate compound, is indicative of the efficacy of the candidate compound with regard to said disease.

With the characteristic being, in one of the deformation classes, in particular in the class of deformed nuclei or preferentially in the class of trapped nuclei, the statistical distribution of the or at least one of the morphological parameters of the cells in the first and second sample. The process then preferentially comprises the comparison of said statistical distribution of the first sample with a statistical distribution profile of the second sample, the observation of a statistical difference between the first and second sample, and/or the observation of a non-significant statistical difference between the healthy sample and the sample in the presence of the candidate compound and of a significant statistical difference between the healthy sample and the sample without the candidate compound, being indicative of the efficacy of the candidate compound with regard to said disease.

### BRIEF DESCRIPTION OF THE DRAWINGS

FIG. 1 schematically depicts the overall process for characterizing the deformability of cells or a portion of a cell in a cell sample,

FIG. 2A depicts a sectional detail of a microstructured plate holding cells, the nuclei of which are trapped in the microgrooves,

FIG. 2B depicts a perspective and sectional view along A-A of a microstructured plate holding cells, the nuclei of which are visible in fluorescence,

FIG. 2C depicts a perspective and sectional view along A-A of a microstructured plate holding cells, the bodies of which are visible in fluorescence,

FIG. 3 shows fluorescence images of nuclei, acquired for samples of different cell types,

FIG. 4 is a graph depicting the percentage of trapped nuclei identified as a function of the mean nuclear volume for some of the cell types from FIG. 3,

FIG. 5 depicts the deformation classes and an example of a fluorescence intensity profile corresponding to each deformation class,

FIG. 6 is an acquired fluorescence image on which the nuclei of each deformation class have been identified in different colors,

FIG. 7 shows fluorescence images of myoblast nuclei acquired for microgrooves of different widths,

FIG. 8 is a box plot depicting the percentage of trapped nuclei as a function of the widths from FIG. 7,

FIG. 9 is a box plot depicting the percentage of deformed nuclei as a function of the widths from FIG. 7,

FIG. 10 shows fluorescence images acquired for plates of different depths,

FIG. 11 is a box plot depicting the percentage of trapped nuclei as a function of the depths from FIG. 10,

FIG. 12 is a box plot depicting the percentage of deformed nuclei as a function of the depths from FIG. 10,

FIG. 13 is a box plot depicting the percentage of trapped nuclei for a healthy myoblast sample (WT) and a diseased myoblast sample (MU Lamin A),

FIG. 14 is a box plot depicting the percentage of deformed nuclei for a healthy myoblast sample (WT) and a diseased myoblast sample (MU Lamin A),

FIG. 15 is a box plot depicting the percentage of trapped nuclei for a healthy breast epithelial cell sample (MCF10A) and a diseased breast epithelial cell sample (MCF7),

FIG. 16 depicts the statistical distributions in circularity determined for all the cells of the sample (a), for the cells for which the nuclei are trapped (b), and for the cells for which the nuclei are deformed (c), for a healthy myoblast sample (WT) and a diseased myoblast sample (MU), and

FIG. 17 depicts the statistical distributions in the ratio of the elliptic Fourier coefficients determined for all the cells of the sample (a), for the cells for which the nuclei are trapped (b), and for the cells for which the nuclei are deformed (c), for a healthy myoblast sample (WT) and a diseased myoblast sample (MU).

### DETAILED DESCRIPTION

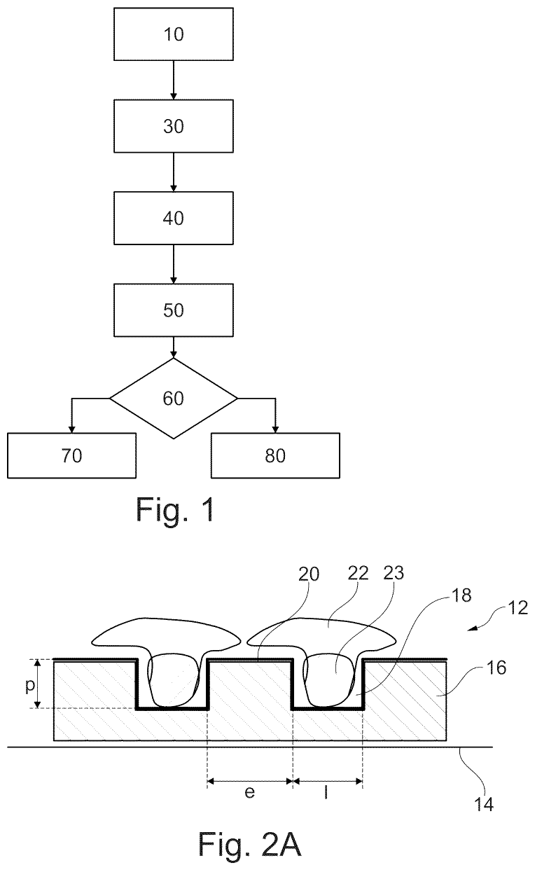

FIG. 1 depicts the overall process for characterizing the deformability of cells or a portion of a cell in a cell sample.

The cell sample can be a sample of adherent cells, comprising muscle cells, endothelial cells, epithelial cells, nerve cells, bone cells, adipose cells, podocytes, cancer cells or a mixture of said cells. The cells can be human, animal or plant cells.

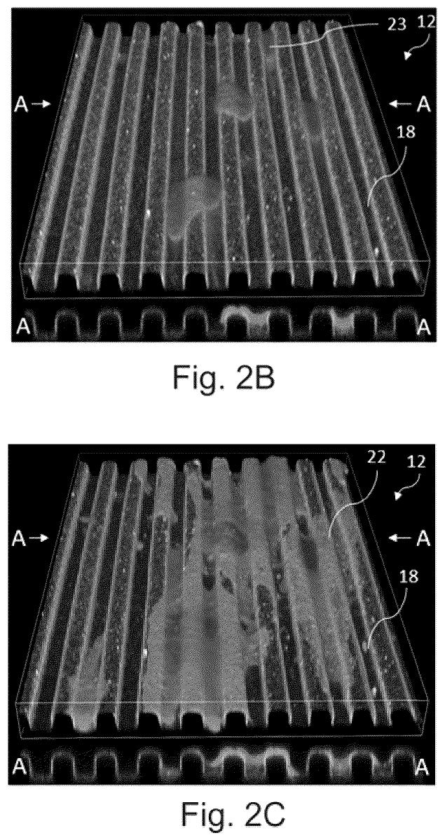

In step 10, the cell sample is deposited on the surface of a microstructured plate 12, illustrated in FIGS. 2A to 2C, and is cultured on the surface of the plate 12 in a suitable medium for a duration of at least 1 h.

The plate 12 comprises a transparent support 14, in particular made of glass, and a microstructured polymer structure 16, in particular made of polydimethylsiloxane (PDMS), secured to the support. The surface of the microstructured structure 16 has a plurality of microgrooves 18, which are preferably of a constant width l and depth p over their entire length, and which are identical, parallel and regularly spaced. However, this could be otherwise: the microgrooves could comprise at least two distinct longitudinal portions, each of a constant width and depth but which differ from one another in terms of their depth and/or width, and/or the microstructured plate could comprise at least two distinct zones each comprising microgrooves which are identical in said zone but which differ between the two zones in terms of the width, depth and/or spacing of the microgrooves.

The microgrooves 18 are of a predetermined width l and depth p so as to enable the at least partial engagement of the nucleus 23 of at least one of said cells in one or more of the microgrooves. They are also preferably dimensioned so as to prevent the whole body of said cells 22 from engaging in a microgroove 18. To this end, the width l and depth p of the microgrooves 18 can be chosen on the basis of at least one physical parameter of cells of the cell type of the sample, in particular their mean dimension or the mean dimension of their nucleus, and/or a biophysical characteristic of these cells, in particular their mean rigidity, mean adhesion strength or mean contractility. The width l and depth p of the microgrooves 18 can be chosen such that the percentage of nuclei entirely trapped in the microgrooves 18 is a parameter indicative of a biological characteristic of the cells of the cell type which is to be determined, in particular of the diseased or non-diseased nature of the cells. For example, the mean percentage of nuclei of healthy or malignant cells of said cell type, in particular in the case of endothelial or muscle cells, which are entirely trapped in the microgrooves, can be between 10% and 90%. The width l, spacing e and depth p of the microgrooves 18 can be between 30 and 60% of the length of the mean minor axis of the nucleus of the non-deformed cells. The width l of the microgrooves can be between 3 and 10 μm, the depth p of the microgrooves can be between 4 and 10 μm, and the spacing e between the microgrooves, measured between the adjacent edges of the microgrooves, can be between 3 and 10 μm.

The surface of the microstructured structure 16 is at least partially treated with a cell adhesion agent, such as fibronectin 20. Such a treatment can be carried out by passing through plasma then incubating in a solution containing fibronectin. The treatment is preferably carried out over the whole surface of the plate 12, but this could be otherwise. It could be carried out only in the bottom of the microgrooves 18 and/or on the side walls.

During the culturing of the cells 22, they deform or do not deform due to the reliefs in the plate 12, and the nuclei 23 of the cells engage to a greater or lesser extent in the microgrooves of the plate 12.

The cells 22 are fixed by adding a fixing compound, for example an aldehyde compound, in particular paraformaldehyde, and the nuclei of the cells are labeled with a fluorescent label such as 4′,6-diamidino-2-phenylindole (DAPI) after the cells have been permeabilized with a permeabilizing agent such as Triton X-100. They could be labeled otherwise, in particular by transduction of the cells with a plasmid encoding a nuclear protein fused to a fluorescent protein, or by adding a fluorescent nuclear label before fixation, such as Hoechst stains. Fixing the cells makes it possible to overcome their dynamism, since confining the nuclei in the microgrooves can statistically be reversed over time.

The cell sample is preferably configured so that the cell density of the cell sample at the moment they are fixed avoids cell confluence. At the moment the cells are fixed, the cell density can be between 10 000 and 50 000 cells/cm².

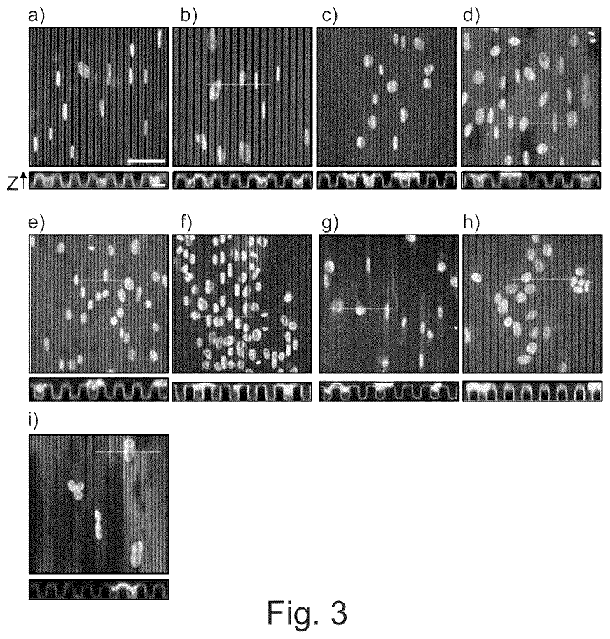

Fluorescence images of the surface of the plate 12 are then taken, in step 30, using a fluorescence microscope. As illustrated in FIG. 3, the nuclei of the cells and their shapes can be identified by the fluorescence they emit. It is clearly apparent from FIG. 3 that this applies to the different cell types mentioned above.

The analysis, preferably automated analysis, of the images obtained in step 40, makes it possible to determine, for each cell nucleus 23 which can be identified, a fluorescence intensity profile along an axis, preferably perpendicular to the longitudinal axis X of the microgrooves and substantially in the middle of the nucleus.

This also makes it possible to determine a contour for each nucleus 23, in particular by adjusting a shape model, for example an ellipse model, in order to deduce different parameters of nuclear shapes therefrom, in particular the following parameters:
roundness, defined by the ratio of the area of the nucleus to the area of a circle circumscribed on the nucleus, giving the following formula:

$$4 * \frac{A}{{π [ M ]}^{2}} ,$$

A being the area of the nucleus and M being the greatest length of the nucleus,
circularity, defined by the ratio of the area of the nucleus to the area of a circle, the radius of which is defined by the length of the contour of the nucleus, giving the following formula:

$$4 π * \frac{A}{{c}^{2}} ,$$

C being the length of the contour of the nucleus,
the aspect ratio, defined by the ratio of the length of the longest axis to the length of the shortest axis of a model ellipse,
solidity, defined as the ratio of the area of the nucleus to the area of the convex nuclear envelope,
the ratio of the elliptic Fourier coefficients, defined by the following formula:

$$\frac{{( {M}_{n} + {m}_{n} )}_{n = 1}}{{\sum }_{2}^{n} ( {M}_{n} + {m}_{n} )} ,$$

Mₙ being the length of the longest axis of the ellipse model of harmonic n, and mₙ being the length of the shortest axis of the ellipse model of harmonic n, and/or
the negative mean curvature, defined as the mean of the concave curvatures of the contour of the nucleus.

The fluorescence intensity profile and the morphological parameter(s) make it possible, in step 50, to classify each cell for which the nucleus 23 can be identified into a deformation class from among a plurality of pre-established deformation classes. In particular, the deformation classes can be based on the percentage of penetration of the nucleus into the microgrooves. The classification is made, for example, from the three classes corresponding respectively to:
i) a freely suspended nucleus, corresponding for example to a substantially flat fluorescence intensity profile or one which is devoid of major fluorescence intensity peaks, associated with one or more morphological parameters characteristic of a nucleus contour that is not very deformed, in particular that is substantially circular, and/or of a low tortuosity of the nucleus contour, for example a high degree of circularity, roundness, solidity and/or ratio of elliptic Fourier coefficients, in particular close to 1, and/or a low aspect ratio and/or negative mean curvature, in particular less than or equal to a respective predetermined threshold value,
ii) a deformed nucleus, extending into at least two adjacent microgrooves, corresponding for example to a fluorescence intensity profile having at least two main peaks, associated with at least one morphological parameter characterizing significant tortuosity of the contour, in particular low solidity compared to a predetermined threshold value, and/or a low ratio of elliptic Fourier coefficients compared to a predetermined threshold value, and/or a high negative mean curvature compared to a predetermined threshold value, and/or low circularity and/or roundness compared to a predetermined threshold value,
iii) a trapped nucleus, in particular entirely inserted in a microgroove, corresponding for example to a fluorescence intensity profile having a main peak, associated with at least one morphological parameter characterizing elongation of the nucleus, in particular low circularity and/or roundness compared to a predetermined threshold value, and/or a high aspect ratio compared to a predetermined threshold value.

FIG. 5 depicts an example fluorescence intensity profile for each of the three classes above. As can be seen on FIG. 5, it is also possible, to aid in classification, to take a confocal measurement for each nucleus, in particular in a mid-plane of the nucleus transversely to the plate and perpendicularly to the grooves. Such a measurement enables clear visualization of the depth of penetration of the nucleus, which may make it possible to refine the classification, in particular during prior study of the relevant parameters. However, such a measurement is not essential.

The class of each nucleus can be reported on the fluorescence image using a color code in order to enable rapid identification directly on the image, as is illustrated in FIG. 6.

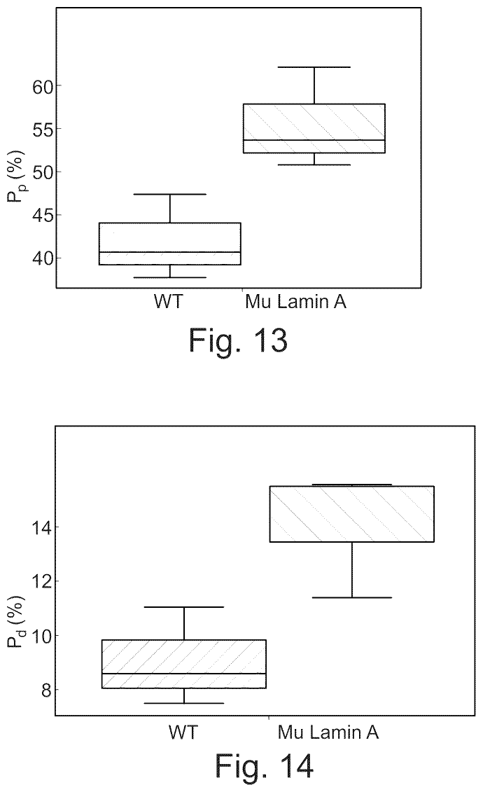

There are then a plurality of possibilities for determining a biological characteristic of the cell sample, in particular the diseased or healthy nature thereof, in step 60. It is possible to compare the percentage of cells of the sample in one of these classes, in particular the class of trapped nuclei or deformed nuclei, to that of another reference sample for which the biological characteristic is known, in particular a healthy sample, as can be seen in FIGS. 13 to 15. It is also possible to compare, in one of the deformation classes, in particular the class of trapped nuclei or the class of deformed nuclei, a statistical distribution of one of the morphological parameters with that of a test sample of cells for which the biological characteristic is known, in particular a healthy sample, as can be seen in FIG. 16 or 17. These different methods can be combined with one another if necessary. The method for determining the biological characteristic depends in particular on the plate used, on the cell type, and on the cell characteristic to be determined. The best method for discrimination can be determined beforehand using statistical tests. These statistical tests take place under the same general conditions (same plate type, same culture method and same imaging method) on samples having biological characteristics which are known to be different, in particular diseased and healthy. These prior statistical tests are preferentially performed on a plurality of panels of reference samples in order to verify the stability of the identification method.

Then, depending on the comparison obtained between the sample to be tested and the reference sample, either it is identified in step 70 that the biological characteristic is identical to that of the reference population, or it is identified in step 80 that the biological characteristic is different to that of a reference population.

Such a study has several possible applications. For example, it makes it possible to determine the diseased or non-diseased nature of a sample from a patient by comparing it to one or more healthy or diseased reference populations in order to deduce a diagnosis therefrom. It can also make it possible to screen a candidate compound by comparing the result on a sample that received the candidate compound with that of a control sample that did not receive it, and optionally by comparing the result of the sample that received the candidate compound and the control sample that did not receive it with that of a reference population, in particular a population considered to be healthy.

### Example 1

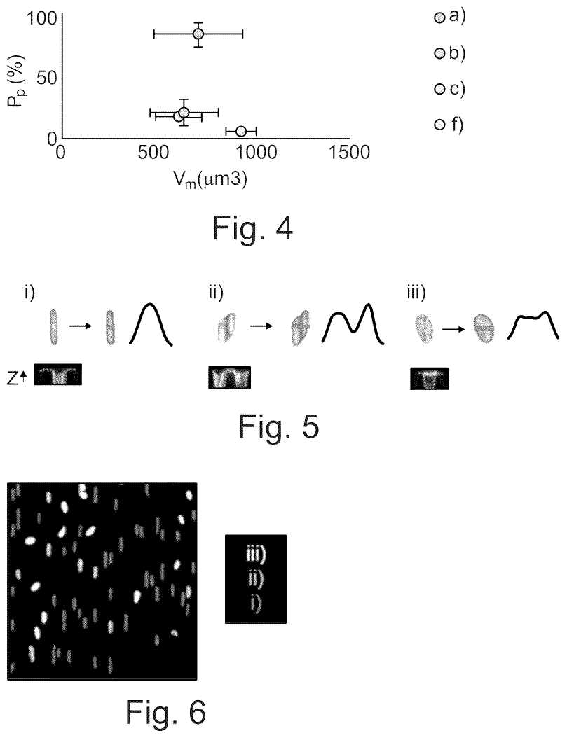

FIGS. 3 and 4 illustrate a study of different cell types.

FIG. 3 shows the images obtained by epifluorescence imaging at the top, and by confocal imaging along the section Z at the bottom, for the following cell types:
a) myoblasts,
b) human umbilical vein endothelial cells (HUVECs)
c) COS-7 cells,
d) HeLa cells,
e) parietal epithelial cells (PECs),
f) breast epithelial cells (MCF-10A),
g) breast cancer cells (MDA-MB-231),
h) breast cancer cells (MCF7), and
i) podocytes.

The cells are cultured on a microstructured plate having parallel microgrooves 5 μm wide and deep, regularly mutually spaced apart by 5 μm.

It can be seen on these images that all the cells studied are able to deform on the plate. The degree of deformation of the nuclei depends on the cell type.

FIG. 4 depicts, for some of these cells, the percentage of nuclei of the cell sample belonging to the class of trapped nuclei Pₚ as a function of the mean nuclear volume Vₘ of the cells (measured in μm on a flat surface) for four of the abovementioned cell types.

It is observed that the percentage of trapped nuclei in the samples Pₚ is not directly dependent on the mean nuclear volume Vₘ. This explains why there is not a simple link between the volume of the nuclei and their deformation.

### Example 2

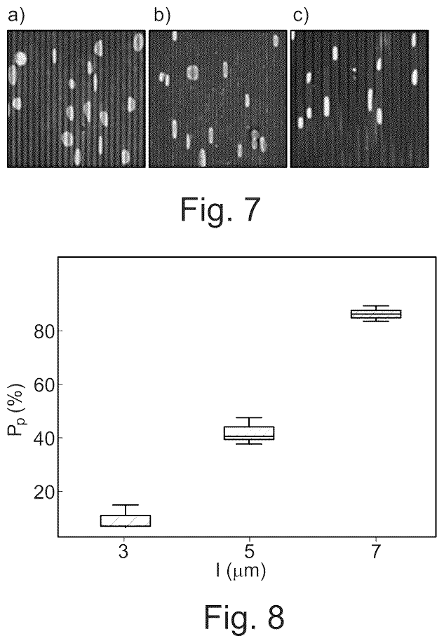

FIGS. 7 to 12 illustrate a study of the influence of the dimensions of the microgrooves on the deformation of the nuclei for myoblast samples.

In this example, the plates are made of microstructured PDMS on a glass support. They are covered over their entire surface with a fibronectin coating. The myoblast cell samples are cultured on the plate in a medium for 8 h. At the end of culture, the cells are fixed using 4% paraformaldehyde for 15 min. After a step of permeabilization using Triton, the nuclei are labeled using a fluorescent label, DAPI, for 1 h. Images of the nuclei are then captured using a fluorescence microscope (20× lens).

FIG. 7 shows the images obtained by epifluorescence imaging for myoblast samples on supports having microgrooves spaced apart by 5 μm, having a depth of 4 μm and being of different widths. The widths are as follows:

$$a ) 1 = 3 μm ,$$

$$b ) 1 = 5 μm , and$$

$$c ) 1 = 7 μm .$$

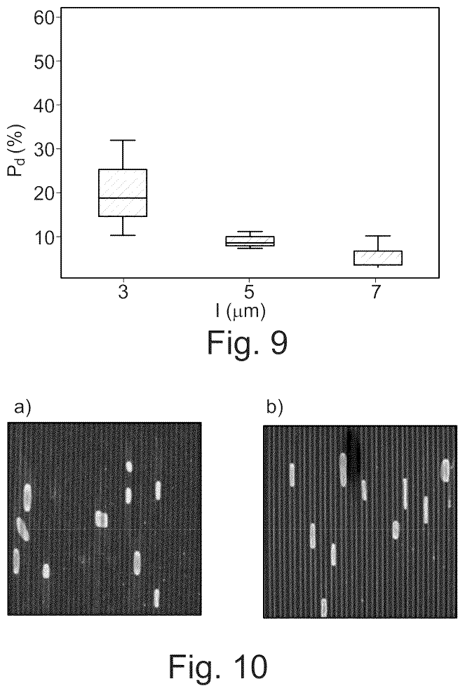

FIGS. 8 and 9 depict, respectively, the percentage of nuclei classified in the class of trapped nuclei Pₚ and the percentage of nuclei classified in the class of deformed nuclei P_{d}, as a function of the width l of the microgrooves. It is observed that increasing the width of the microgrooves changes the proportion of the different classes of nuclei (reduction in percentage of deformed nuclei and increase in percentage of trapped nuclei).

FIG. 10 shows the images obtained by epifluorescence imaging for myoblast samples on supports having microgrooves spaced apart by 5 μm, having a width of 5 μm and being of different depths. The depths are as follows:

$$a ) p = 4 μm , and$$

$$b ) p = 5.4 μm .$$

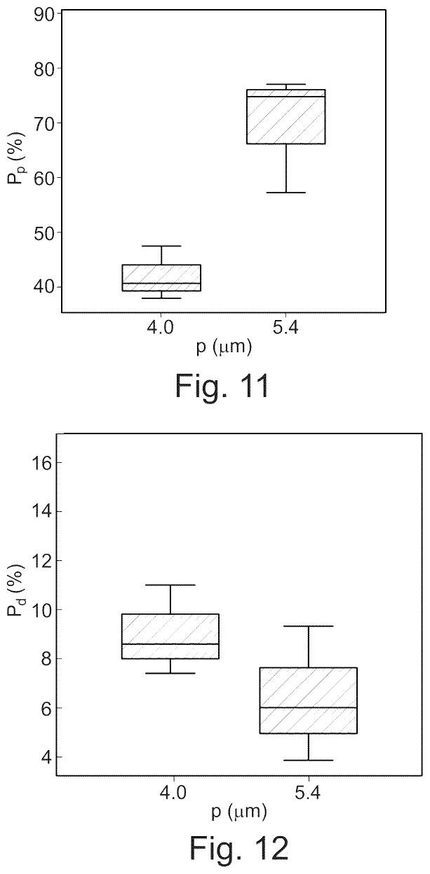

FIGS. 11 and 12 depict, respectively, the percentage of nuclei classified in the class of trapped nuclei Pₚ and the percentage of nuclei classified in the class of deformed nuclei P_{d}, as a function of the depth p of the microgrooves. It is observed that increasing the depth of the microgrooves changes the proportion of the different classes of nuclei (reduction in percentage of deformed nuclei and increase in percentage of trapped nuclei).

Thus, the dimension of the microgrooves has a significant influence on the deformation of the cells on the microstructured plate. The dimensions of the microgrooves can thus be adapted for each cell type on the basis of their deformation potential, in particular by prior study, in order to optimize the study of cell deformation.

### Example 3

FIGS. 13 and 14 depict, respectively, the percentage of trapped nuclei Pₚ and the percentage of deformed nuclei P_{d} for samples of cells of myoblast type from healthy patients (WT) and for samples of cells of myoblast type from unwell patients having a mutation in the gene encoding lamin A, the main component of the nuclear envelope (Mu Lamin A Delta K32). The cell samples are cultured using the method described in the previous example, on a plate having microgrooves of a width 1 of 5 μm, a depth of 4 μm and being spaced apart by 5 μm.

It is observed that the minima and maxima of the percentages of trapped nuclei Pₚ and deformed nuclei P_{d} do not intersect between the healthy samples WT and the diseased samples Mu Lamin A, indicating that there is a significant difference in these parameters between the healthy samples WT and the diseases samples Mu Lamin A. This result shows that determining the percentage of trapped nuclei Pₚ or deformed nuclei P_{d} makes it possible to discriminate between the WT and Mu Lamin A samples, and therefore to detect disease.

### Example 4

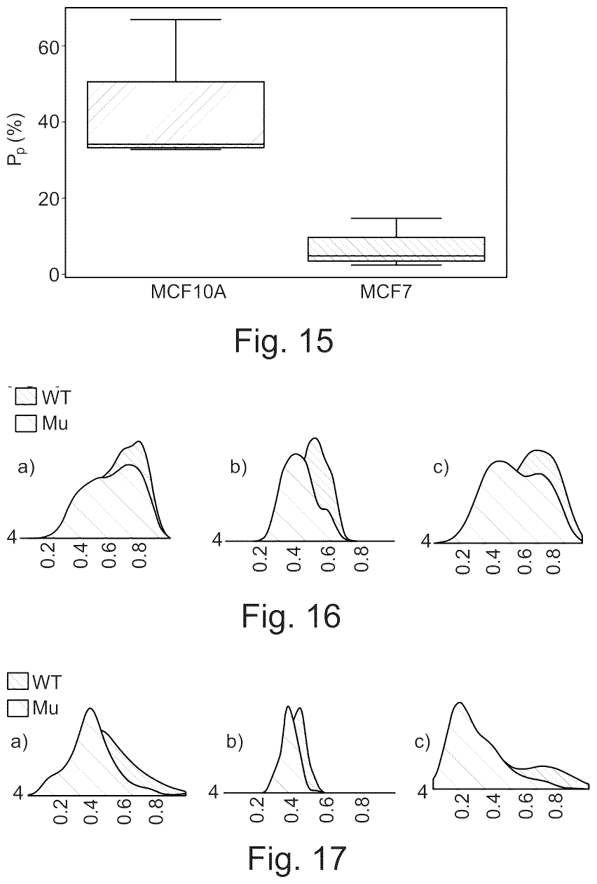

FIG. 15 depicts the percentages of trapped nuclei Pₚ for a sample of healthy breast epithelial cells (MCF10A) and for a sample of cancerous breast epithelial cells (MCF7). The cell samples are cultured for 24 h using the method described in the previous example, on a plate having microgrooves of a width l of 5 μm, a depth of 7.5 μm and being spaced apart by 5 μm.

A significant difference is clearly observed in the percentage of trapped nuclei Pₚ between the sample of non-cancerous breast epithelial cells MCF10A and the sample of cancerous breast epithelial cells MCF7. This result shows that the percentage of trapped nuclei is also a good discriminator in the case of breast epithelial cells.

### Example 5

FIG. 16 depicts the normalized statistical distributions of circularity for all the cells in sample (a), trapped nuclei only (b) and deformed nuclei only (c) for a sample of cells of myoblast type from a healthy patient (WT) and for a sample of cells of myoblast type from an unwell patient having a Lamin A mutation (Mu). The cell samples are cultured using the method described in the previous example, on a plate having microgrooves of a width of 5 μm, a depth of 4 μm and being spaced apart by 5 μm.

The difference d between the maxima of the two distributions, WT and Mu, is calculated to determine the separation in the distributions and to thereby characterize the potential for discrimination between the two cell samples. The difference d is equal to 0.06 in case (a) (all cells), 0.25 in case (b) (trapped nuclei), 0.23 in case (c) (deformed nuclei).

It is observed that the difference d is much larger in cases (b) and (c), confirming that the system of classification prior to the morphological study of the nuclei improves discrimination between the two cell samples WT and Mu.

FIG. 17 depicts the same study, applied to the elliptic Fourier coefficients. The difference d is equal to 0.12 in case (a) (all cells), 0.31 in case (b) (trapped nuclei), 0.12 in case (c) (deformed nuclei).

It is observed that the difference d is much larger in cases (b), confirming that the system of classification prior to the morphological study of the nuclei improves discrimination between the two cell samples WT and Mu.

However, this time only the class of trapped nuclei enables better discrimination. This confirms that prior study can be useful for determining the correct combination of classes and morphological parameters to be studied in order to have optimal discrimination of samples having different biological characteristics.

The invention is not limited to the example described above. For example, the microstructured plate can have microgrooves with a more complex distribution than being regularly spaced apart. The plate could comprise a plurality of zones having different microgroove characteristics.

As a variant, other morphological parameters characterizing the three-dimensional shape of the nucleus can be envisaged if they enable effective discrimination between two cell samples having different biological characteristics.

As a variant, the invention is not limited to the cell and disease types mentioned. The process mentioned has potential for numerous cell types and numerous diseases.

1. A process for characterizing the deformability of cells or a portion of cells in a cell sample, said cells each comprising a body and a nucleus, the process comprising:
culturing said cells on a microstructured plate which has, at the surface, a plurality of microgrooves, the microgrooves being of a predetermined width l and depth p so as to enable the at least partial engagement of the nucleus of at least one of said cells in one or more of the microgrooves, at least part of the surface of the microgrooves being an adhesion surface for the cells,
measuring, by microscopy, a fluorescence signal of the nucleus of said cells, the nucleus of said cells having been treated beforehand to emit fluorescence radiation,
based on the fluorescence signal measured for each nucleus, determining a fluorescence intensity profile for each nucleus along at least one axis of said nucleus, and at least one morphological parameter of said nucleus,
based on the fluorescence intensity profile and on the at least one morphological parameter determined for each nucleus, determining a deformation class of said nucleus in the depth of one or more microgrooves.

2. The process as claimed in claim 1, wherein at least part of the inner surface of the microgrooves, in particular the side walls and/or the bottom of the microgrooves, is coated with an adhesion coating, in particular a cell adhesion protein, for example fibronectin, collagen, laminin or gelatin.

3. The process as claimed in claim 1, wherein the cells are selected from adherent cells, for instance muscle cells, endothelial cells, epithelial cells, podocytes and/or cancer cells.

4. The process as claimed in claim 1, wherein the microscopy measurement comprises acquiring a fluorescence image of the surface of the microstructured plate.

5. The process as claimed in claim 4, wherein the determination of the morphological parameter(s) can comprise the detection of the contour of the nucleus of the cells on the acquired fluorescence image(s), and the morphometric analysis of said shape of the nucleus in order to deduce the morphological parameter(s) therefrom.

6. The process as claimed in claim 1, wherein the microscopy measurement comprises acquiring fluorescence images in a plane and/or a plurality of planes transverse to the microgrooves, in particular by confocal microscopy.

7. The process as claimed in claim 1, wherein the fluorescence intensity profile is determined on the fluorescence signal, in particular on the acquired fluorescence image, perpendicularly to the axis of extension of the microgrooves, preferably in a substantially mid-plane of said nucleus.

8. The process as claimed in claim 1, wherein the at least one morphological parameter of the nucleus of the cells is selected from circularity, roundness, solidity, aspect ratio, elliptic Fourier coefficient, and/or the tortuosity of the nucleus.

9. The process as claimed in claim 1, wherein the morphological parameter(s) are selected such that, for a distribution of the or each morphological parameter, a statistical difference is established between test samples having different biological characteristics, in particular healthy and diseased.

10. The process as claimed in claim 1, wherein the process comprises, based on the fluorescence intensity profile and on the at least one morphological parameter which are determined for each nucleus, determining the deformation class of said nucleus in the depth of one or more microgrooves from at least three predetermined deformation classes which correspond, respectively, to:
a) a freely suspended nucleus,
b) a deformed nucleus, extending at least partially into at least two adjacent microgrooves,
c) a trapped nucleus.

11. The process as claimed in claim 1, comprising determining the proportion of cells in the cell sample in at least one of the deformation classes, in particular the class of trapped nuclei and/or deformed nuclei, and determining a biological characteristic of the cell sample by comparing the proportion of cells in the cell sample which are in the or each deformation class with a reference proportion in said at least one deformation class from a test sample of the same cell type, determined beforehand, in order to determine a biological characteristic of the cell sample, the biological characteristic of the test sample being known.

12. The process as claimed in claim 1, comprising determining a statistical distribution of the or at least one of the morphological parameters of the nuclei of the cells from the sample which are classified in one of the deformation classes, in particular for the class of deformed nuclei and/or the class of trapped nuclei, and comparing a variable of said statistical distribution with the same variable of statistical distribution of the morphological parameter of cells which are classified in said same deformation class(es) in a reference sample for which a biological characteristic is known.

13. A method for diagnosing a disease state in an individual, the method comprising at least the following steps:
a) culturing a sample of a type of cells isolated from said individual, the cells of the sample comprising a body and a nucleus and being cultured on a microstructured plate having, at the surface, a plurality of microgrooves, at least part of the surface of the microgrooves being an adhesion surface for the cells, the microgrooves being of a predetermined width and depth so as to enable the at least partial engagement of the nucleus of at least one of said cells in one or more of the microgrooves,
b) measuring, by microscopy, a fluorescence signal of the nuclei of the cells from the sample, the nuclei of the cells having been configured beforehand to emit fluorescence radiation,
c) based on the fluorescence signal measured for each nucleus, determining a fluorescence intensity profile for each nucleus along at least one axis of said nucleus, and at least one corresponding morphological parameter of said nucleus,
d) based on the fluorescence intensity profile and on at least one morphological parameter determined for each nucleus, determining a deformation class of said nucleus in the depth of one or more microgrooves,
e) comparing at least one characteristic of at least one deformation class of the nuclei of the cells from the sample, obtained in step d), with the same characteristic for classes of a test sample, in order to reach a conclusion therefrom regarding the disease or non-disease state of the cells.

14. A method for screening a candidate compound for the treatment and/or prevention of a disease state, the method comprising at least the following steps:
a) in vitro culture of a first sample of a cell type representative of a disease in the absence of the candidate compound,
b) in vitro culture of a second sample of said cell type representative of said disease in the presence of the candidate compound,
the cells of the first and second samples comprising a body and a nucleus and being cultured on a first and a second identical microstructured plate each having, at the surface, a plurality of microgrooves, at least part of the surface of the microgrooves being an adhesion surface for said cells, the microgrooves being of a predetermined width and depth so as to enable the at least partial engagement of the nucleus of at least one of said cells in one or more of the microgrooves,
c) measuring, by microscopy, a fluorescence signal of the nuclei of the cells from the first and second samples, the nuclei of the cells from the first and second samples having been configured beforehand to emit fluorescence radiation,
d) determining fluorescence intensity profiles along at least one axis of the nucleus, and at least one morphological parameter of the nucleus of each cell from the first and second samples, based on the respective measured fluorescence signals,
e) determining deformation classes of the nucleus of each cell from the first and second samples in the direction of the depth of the microgrooves, based on the determined fluorescence intensity profile and on at least one determined morphological parameter, and
f) comparing at least one characteristic of the first sample in at least one deformation class of the nuclei of the cells from the first sample with the same characteristic for the second sample, the observation of a difference between said characteristics of the first and second sample being indicative of the efficacy of the candidate compound with respect to said disease.

15. The process as claimed in claim 14, comprising the culture of a third sample of said cell type which is considered to be healthy in the absence of the candidate compound; measuring, by microscopy, a fluorescence signal of the nuclei of the cells from the third sample, the nuclei of the cells of the third sample having been configured beforehand to emit fluorescence radiation; determining the fluorescence intensity profile along at least one axis of the nucleus, and at least one morphological parameter of the nucleus of each cell from the third sample, based on the respective measured fluorescence signals; determining deformation classes of the nucleus of each cell from the third sample in the direction of the depth of the microgrooves, based on the determined fluorescence intensity profile and on at least one determined morphological parameter; and comparing at least one characteristic of the first sample and/or of the second sample in at least one deformation class of the nuclei of the cells from the first sample and/or from the second sample with the same characteristic for the third sample, the observation of a difference between said characteristics of the first and third sample and/or a similarity between said characteristics of the second and third sample being indicative of the efficacy of the candidate compound with respect to said disease.
